(19) United States
(12) Patent Application Publication
(10) Pub. No.: US 2012/0258431 A1
(43) Pub. Date: Oct. 11, 2012

(54) Method and System for Tracking Jaw Motion

(76) Inventor: Mark D. Lauren, Amherst, NY (US)

(21) Appl. No.: 13/372,110

(22) Filed: Feb. 13, 2012

Related U.S. Application Data
(60) Provisional application No. 61/516,868, filed on Apr. 11, 2011.

(51) Int. Cl. A61B 5/11; A61C 13/00

(52) U.S. Cl. 433/201.1; 600/595

(57) ABSTRACT

Methods are provided to record and utilize jaw motion data using low-angle photogrammetric techniques. A method is described based on using an extraoral imaging apparatus to capture images of texture placed on tooth or soft tissue surfaces. Upper and lower textural surface regions of the oral cavity are imaged simultaneously, and their surfaces derived as a function of time to produce 4d data. The clinically derived surface data may be directly registered to the 3d anatomy of an individual, providing the ability to animate the relative motion of the mandible. An imaging apparatus suitable for capture of low-angle image groups is disclosed.

### CROSS-REFERENCE TO RELATED APPLICATION

This application claims priority to U.S. Provisional Application No. 61/516,868, filed on Apr. 11, 2011, now pending, the disclosure of which is incorporated herein by reference.

### FIELD OF THE INVENTION

The invention relates to the art of capturing and modeling jaw motion, and more particularly to generating a 4-dimensional (“4d”) model of a person's jaw.

### BACKGROUND OF THE INVENTION

Many methods have been disclosed to record human jaw movement, including mechanical, electronic, ultrasonic, electromagnetic, and optical techniques. Modern commercial systems use physical frames separately mounted to the maxilla and mandible, with the relative 3-dimensional (“3d”) position of the frames being detected and recorded. Frame-based systems are far from ideal, being cumbersome, time-consuming to setup, and have limited accuracy. The presence of the frames also inherently disrupts an individual's natural jaw movement.

Photogrammetry has been used to extraorally measure jaw movement without the use of frames. Using target-based photogrammetry, the 3d location of a relatively small number of targets, somehow attached to the teeth and positioned outside the mouth, has been determined. Generally, a mouth-piece or plastic attachment is used to hold the imaging targets stationary with respect to the mandible and maxilla.

Baumrind (U.S. Pat. No. 4,836,778) discloses a frame-based target system using light-emitting diodes as targets at the vertices of triangular fixtures held outside the mouth by support elements bonded to the teeth.

Neumeyer (U.S. Pat. No. 4,859,181) describes a photogrammetry system that uses plastic ‘holding elements’ attached to the teeth to position ‘reference elements’ containing targets outside the mouth.

Robertson (U.S. Pat. No. 5,340,309) uses cuboidal targets with crosshairs held outside the mouth.

Baba (U.S. Pat. No. 5,905,658) places ‘measurement points’ on the dentition without specifying a structure or method for attaching the ‘jaw movement measuring points’ to the teeth.

Lauren (U.S. Pat. Appl. Pub. No. 2010/0198566) (“Lauren”) teaches a fluorescent method using targets applied to tooth surface. The 3d target locations are obtained using an extraoral imaging device.

The practical applications of jaw motion data include improved dental prosthesis design, characterization and analysis of the motion, and integration with alternate imaging modalities, such as x-ray, to provide enhanced biomedical imaging. For prosthesis design, jaw motion data may be used to animate the anatomy used; in this way, the design can account for the patent-specific movement of the jaw. This requires the captured jaw motion data to be registered to the required 3d design anatomy.

This registration process involves matching the 3d point clouds of the clinically captured surface with the 3d design anatomy. Since the clinically captured surface is only a fraction of the anatomy to be animated, accurate registration is critical to the successful commercial application of these methods.

From the perspective of producing 3d surface data suitable for registration to design anatomy, prior art jaw motion methods have specific limitations.

In the Lauren method, the brushing of imaging elements onto the teeth results in a non-uniform distribution of targets on the surface. While a polygon surface may be constructed using individual target locations, the 3d point density is uncontrolled. This results in two problems: 1) incomplete surface modeling, and 2) poor registration to design anatomy.

1) Surface regions that do not contain targets result in gaps, or missing sections of the surface. This makes it impossible to produce a complete and accurate representation of the surface. While smoothing approximations may be applied to fill-in surface gaps, this generally leads to additional error.

2) Non-uniform target spacing results in a non-regular 3d point mesh. Non-uniform fields of 3d point data are difficult or impossible to accurately register to the uniform point cloud data routinely produced for the design anatomy. Point-based surface registration methods work best between surfaces having similar point mesh densities.

Target-based methods also require imaging to take place at high angles, providing increased accuracy as the angle between camera images approaches 90°. This requires the cameras to be spaces far apart from each other which generally leads to a larger and unbalanced design that is not conveniently hand-held. Accordingly, there also is a need for a more compact imaging unit.

### BRIEF SUMMARY OF THE INVENTION

The methods of this invention overcome these limitations using a low angle imaging approach which provides: 1) the ability to tolerate surface gaps of the imaging elements, 2) a uniform point mesh for accurate registration, and 3) a practical hand-held imaging device.

While imaging elements may still be non-uniformly applied by brushing, small regions without imaging elements may be tolerated. Pixels on the camera detector that correspond to surface regions without imaging elements still receive a detectable amount of light from surrounding imaging elements. While not suitable for determining target locations, such low-level light data does provide texture information useful for 3d modeling according to the invention.

Instead of deriving individual target locations to define a 3d surface, software algorithms are used to recognize and correlate textural patterns between pairs of images taken at low angles to derive a uniform point mesh of controlled density. The uniform point mesh, produced with minimal gaps in the surface data, facilitates accurate registration to the design anatomy due to the similarity in point mesh density.

The low angle camera configuration of this invention also provides for a compact extraoral hand-held imaging device, suitable for use in the dental clinic.

The present invention describes methods to conveniently capture and integrate 4-dimensional (“4d”) motion data into dental computed aided design (CAD).

Random patterns of microscopic imaging elements are applied to regions of both the upper and lower teeth or soft tissues of the mouth in the form of patches which may extend over several teeth. The imaging elements provide a randomized optical pattern, referred to as ‘surface texture’, on the sensor of an imaging device. The texture can vary spatially and in intensity.

In a preferred embodiment, fluorescent microspheres are used to provide texture. The microspheres are held on the surface by a thin colored film that also provides a dark background between the imaging elements. Incident blue light causes the microspheres to fluoresce green. An optical bandpass filter in front of a monochrome camera allows the fluorescing texture to be imaged as white against a dark background. At the same time, the blue excitation light is blocked from the camera sensor.

The upper and lower textural regions (patches) are imaged from outside the mouth using a hand-held rig that contains three cameras fixed at low angles to each other. The cameras image simultaneously at a controlled rate of about 10 Hz, with a triplet of images being produced at each time point.

The 3d surfaces of the upper and lower patches are derived from each triplet (or group) of images and saved as a surface patch file. The methods of this invention use image-based registration methods on low angle image pairs to derive a uniform 3d point mesh for each group of images. These methods use software algorithms to recognize and correlate textural patterns between pairs of images to derive a uniform point mesh of controlled density.

Surface patch files, containing both upper and lower textured patch surfaces, represent an accurate 3d record of the relative position of the upper and lower arches at a particular time point. A time-based set of 3d surface patch files is produced from the corresponding set of image triplets captured during a jaw motion sequence. This is the basic data produced by the method of the invention. These data may then be used to derive a 4d model and integrated into CAD software.

Clinically, image capture takes place while an individual performs specific jaw motions. For example, protrusion motion data may be used to assist with designing the anterior guidance required for proper posterior disclusion. Also, basic open/close motion data may be used to derive a true arc-of-closure of an individual which can assist with ensuring that cusp tips enter the fossa of opposing teeth properly and contact the bottom of the fossa in a balanced way.

The integration of time-based surface patch data into CAD may be achieved by a number of methods well-known in the art. This generally requires the dynamically captured surface patch data to be registered to anatomy produced by digitizing a dental cast or intra-oral scanning.

For example, one of the surface patch files in a sequence may be defined as a reference position for relative motion modeling. The upper and lower design anatomy may then be registered to this reference position. Since the surface patch files have a uniform point density, they are readily registered to the digitized design anatomy. The upper patch from another (second) surface patch file in a sequence may then be registered to the upper reference position. The lower patch in the second surface patch file is displaced from the lower reference position. Registering the lower reference position to the second lower surface patch provides a transform to describe the change in position of the lower anatomy from its position in the reference to its position in the second surface patch file. Continuing this process for each surface patch file in a sequence produces a set of transforms to describe the incremental change in position of the lower arch, with a fixed upper position. These transform data represent a 4d model and are readily integrated in to CAD to provide the desired animation.

An object of this invention is to provide a convenient jaw tracking method that produces dynamic 3d data that may be readily integrated into dental CAD systems.

Another object of the invention is to provide a composition and associated application method for fluorescent microsphere imaging elements to be applied to the teeth to provide the texture needed for imaging.

A further object of the invention is to provide an extraoral hand-held imaging device, suitable for use in the dental clinic, for performing extraoral jaw tracking imaging. Since the cameras are positioned close together for the low angle imaging, a compact imaging device is provided.

The methods are non-invasive, clinically practical, and do not requiring any mechanical fixtures to be attached to the individual.

### DESCRIPTION OF THE DRAWINGS

For a fuller understanding of the nature and objects of the invention, reference should be made to the following detailed description taken in conjunction with the accompanying drawings, in which:

FIG. 1A is a flowchart showing a method according to an embodiment of the present invention for producing 4d jaw motion data.

FIG. 1B is a flowchart showing a method according to another embodiment of the present invention for designing dental prosthetics.

FIG. 2 is a flowchart showing an exemplary image-based registration method.

FIG. 3 is a flowchart showing an exemplary method of applying different textures to the upper and lower arches.

FIG. 4 is a flowchart showing an exemplary method for modeling and utilizing the 4d jaw motion data produced by the present invention.

FIG. 5 shows the approximate field of view of an extraoral apparatus. The location of upper and lower texture patches on the teeth and soft tissue is shown.

FIG. 6 shows a single image of fluorescent imaging elements in the mouth of an individual and captured by a monochrome imaging device. The microsphere texture images as white against a dark background.

FIG. 7 shows a 3d surface patch file derived from an image group, for a single time point.

FIG. 8 shows the main elements of the low-angle 3-camera configuration of an exemplary imaging apparatus according to the present invention.

FIG. 9 shows the arrangement of functional components of the imaging apparatus of FIG. 8.

FIG. 10 depicts an imaging apparatus according to another embodiment of the present invention.

### DETAILED DESCRIPTION OF THE INVENTION

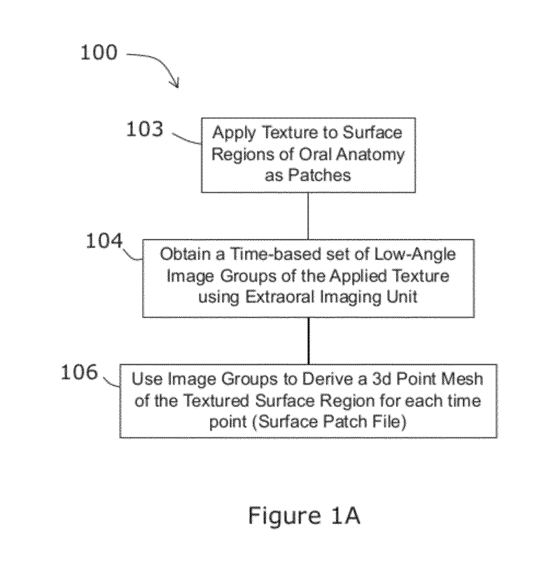

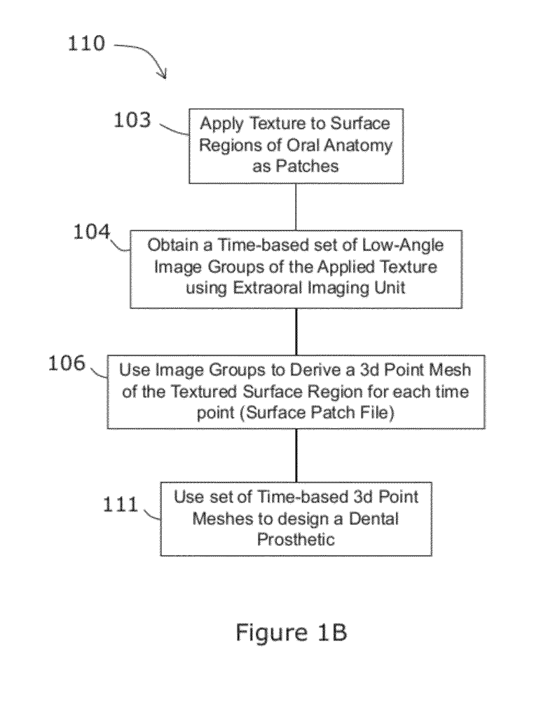

The present invention may be embodied as a method 100 for tracking jaw motion (see, e.g., FIG. 1A). Tracking jaw motion may be accomplished by capturing and recording the 4-dimensional (“4d”) movement of the jaw. As used herein, 4d systems are 3-dimensional (“3d”) systems that change with time, with time being the fourth dimension.

The jaw of an individual has an upper arch and a lower arch. Each arch may contain soft tissue and a plurality of teeth. A texture is applied 103 to one or more surface regions of the upper arch and one or more surface regions of the lower arch. The surface regions with applied texture may be referred to as a “textured surface region” or a “patch.” For the purposes of the present disclosure, a texture is any treatment applied to a surface which results in a generally random optical pattern capable of being detected by an image sensor. Examples of textures include, but are not limited to, fluorescent or colored polymer microspheres, luminescent materials, printed patterns transferred to the mouth, and texture-providing components such as sand, or projected optical patterns. Typical fluorophores used with textures include, without limitation, fluoresein, fluorescein isothiocyanate, and Nile red.

In an exemplary embodiment, a texture, comprising fluorescent polymer microspheres (such as those available from Polysciences, Inc., of Warrington, Pa.), is applied 103 to the surface region(s). The microspheres may be between approximately 5 and 100 microns in diameter. The surface region of each arch may be a tooth surface, a soft tissue surface, or a combination of tooth and soft tissue surfaces.

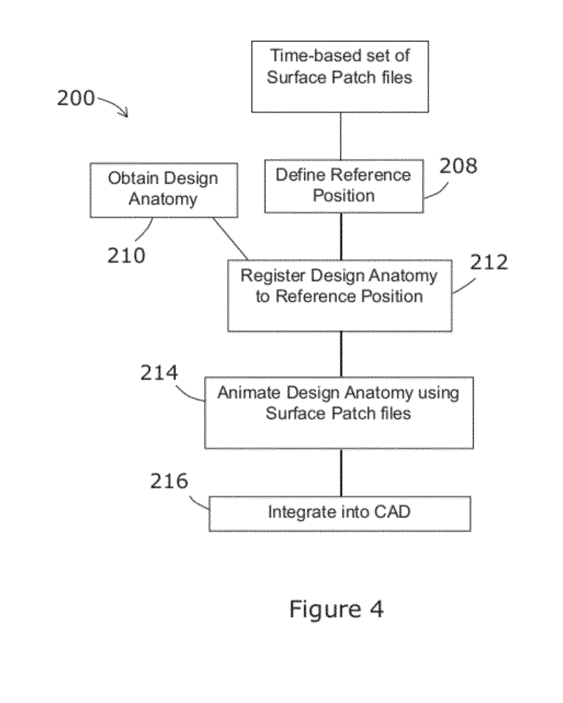

FIG. 5 depicts an example wherein texture is applied 103 to the surface regions 340 of the upper arch and surface regions 360 of the lower arch, within the field of view 320 of an imaging apparatus. Surface regions are selected based on factors such as: 1) being within the anatomy to be animated, 2) having curvature, and 3) being readily accessible for imaging.

The texture may be applied 103 to the surface regions as a liquid composition. Such compositions may include a carrier substance such as, for example, an alcohol. The method 100 may comprise the step of waiting for the texture to dry and/or cure. Such liquid compositions may be applied by brushing (or painting-on), spraying, or the like. The texture may be configured as a solid, for example, a granular composition. The texture may take other formats including a water-transfer decal. Such decals are typically purchased as a (dry) decal on a transfer substrate (e.g., paper, etc.). The decal is then transferred off of the substrate and onto the surface region of the arch using water and/or a decal adhesive. The decal adhesive may be water-soluble.

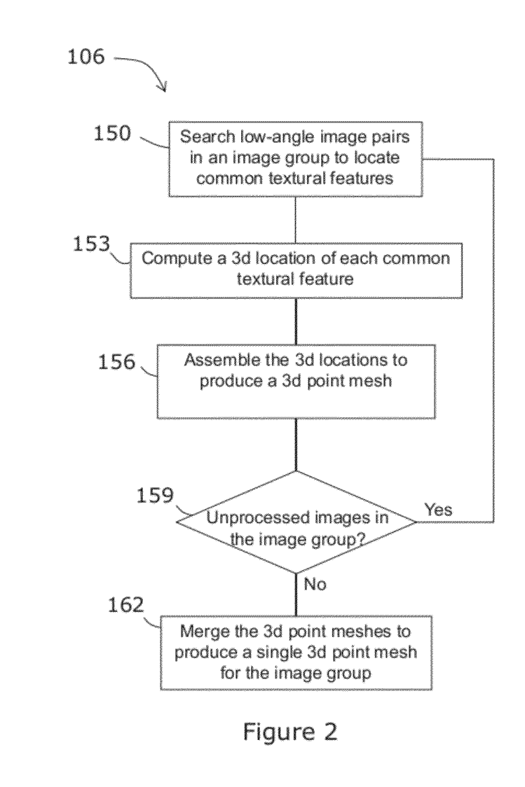

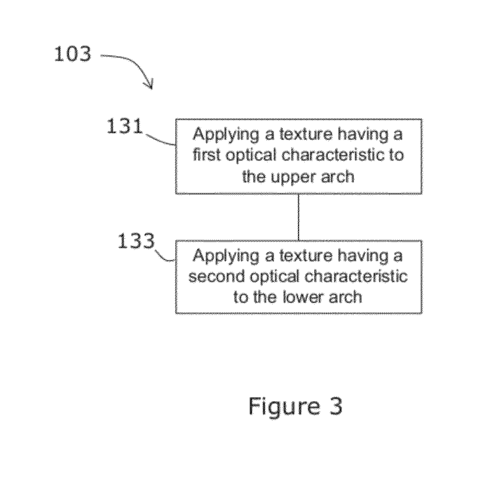

The texture applied 131 to the surface region(s) of the upper arch may have a different optical characteristic than the texture applied 133 to the surface region(s) of the lower arch (see, e.g., FIG. 3). For example, the upper arch texture may have a different color than the lower arch texture.

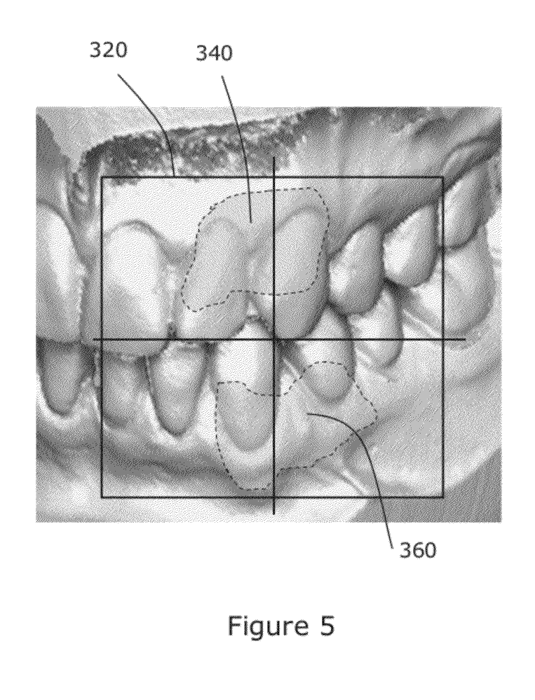

In an exemplary embodiment, fluorescent microspheres are used to provide texture, where the microspheres emit green light when excited by blue light. In such an embodiment, a green optical bandpass filter in front of a monochrome digital camera allows the texture to be detected by the camera as white against a dark background. FIG. 6 shows an image, obtained by such a camera, of upper 404 and lower 406 patches (i.e., with applied texture), and an adjacent tooth 402, without an applied texture, which is seen to fluoresce in response to incident blue light.

The method 100 further comprises obtaining 104 a time-based set of image groups wherein each image group comprises at least two images of a region of interest taken simultaneously at low angles. The images of each image group are obtained 104 simultaneously and each image of each image group is obtained 104 along an optical axis which is less than 30° with respect to an optical axis used to obtain another image within the same image group. As such, the images of each image group are said to be at “low angle” to one another.

In an embodiment, each image group comprises three images (a “triplet” of images or an “image triplet”). For example, the triplet of images may be obtained 104 from three cameras positioned at fixed, low angles to each other (as further described below). The cameras are triggered so as to obtain 104 the images of the image group (triplet) simultaneously.

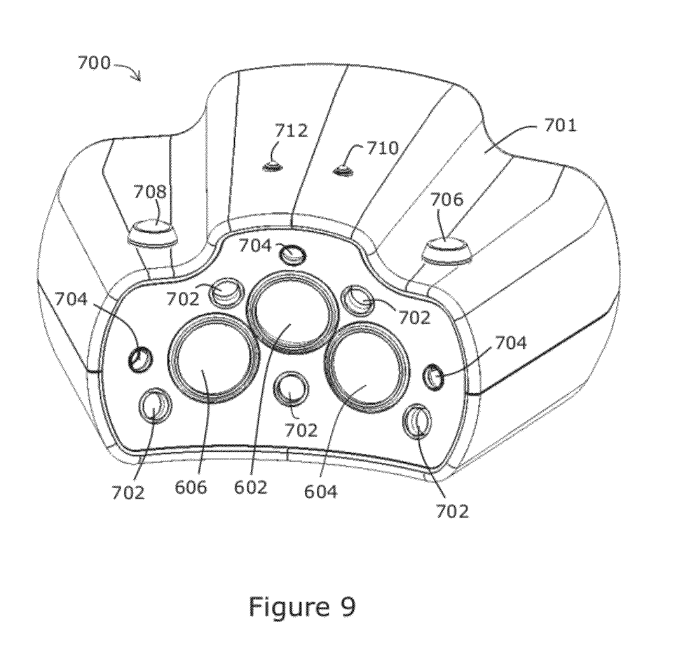

Each image group is obtained 104 using an imaging apparatus, such as, for example, the imaging apparatus 700 shown in FIG. 9 (having a camera configuration suitable for obtaining an image triplet).

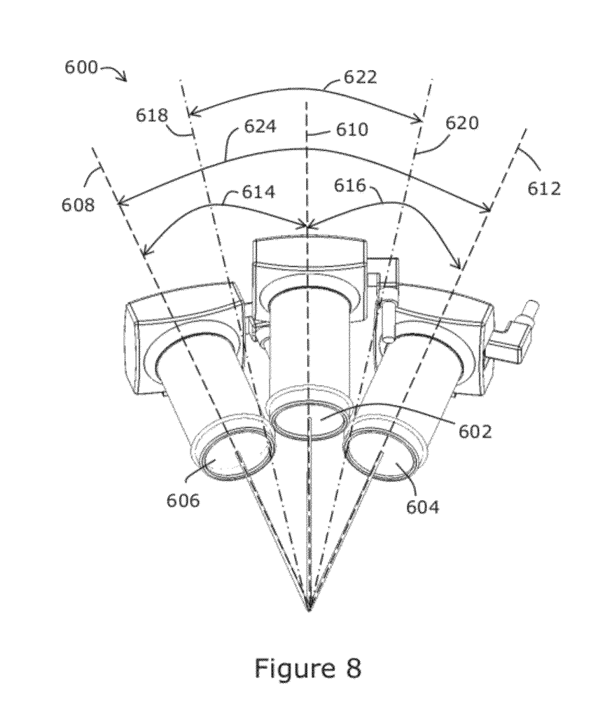

In an exemplary embodiment, each image triplet comprises three images which may be considered as two low-angle image pairs (where one image of the two image pairs is common between the pairs). The two image pairs are obtained 104 using three digital cameras positioned at fixed low angles to each other, for example, as shown in FIG. 8. The cameras obtain the images of an image group simultaneously, while obtaining image groups over time, thereby obtaining 104 an image triplet for each time increment. In such an embodiment, two low-angle image pairs are utilized for image-based registration as further described below; between cameras 602 and 604 as well as cameras 602 and 606. The angle 624 between cameras 604 and 606, in this exemplary embodiment, is too large to be used.

The time-based set of image groups is obtained 104 during the motion of the jaw such that the relative arch positions captured by each image group vary according to the jaw motion. It should be noted that the term, “position,” should be interpreted broadly throughout this disclosure to refer to the relative location of an object in space as well as the orientation of the object. The imaging apparatus may capture image groups at a regular frequency—a “sampling rate.” The sampling rate of an exemplary imaging apparatus may be 5-20 Hz or higher. In an exemplary embodiment, the sampling rate is approximately 10 Hz. The intervals between samples (image groups) need not be regular.

The method 100 further comprises using 106 image-based registration to produce at least two 3-dimensional point meshes of the surface regions having texture. Typically, one 3-dimensional point mesh will be produced for each low-angle image pair.

Using image-based registration methods, when a common textural feature is found (located) 150 in the pair of images, a 3d location is computed 153 for that common textural feature. The 3d location is calculated by using a priori information about the imaging apparatus, including, for example, the position and orientation of the imaging devices with respect to each other. Here again, further information is provided below. The 3d locations of the common textural features, which may be recorded as a set of coordinates for each common textural feature, are assembled 156 into an electronic file to produce the 3-dimensional point mesh for an image pair. The file comprises a plurality of coordinate locations representing the textured surface regions of the arches. This process is repeated 159 for each of the image groups such that a 3-dimensional point mesh is produced for each image group. In this way, a 3-dimensional point mesh is produced for the various relative arch positions recorded in the time-based set of image groups. The meshes produced by low-angle image pairs within each image group are combined (merged) 162 into a single point mesh and may be saved as a surface patch file. Time-based 3-dimensional point meshes together define a 4d model of the positions of the upper and lower arch throughout the jaw motion of the individual. In this way, the point meshes may be said to represent the loci of 3-dimensional positions travelled by the surfaces of the arches during jaw motion. The produced series of time-based 3-dimensional point meshes may be considered to be a 4-dimensional (4d) model of jaw motion.

A time-based set of surface patch files is produced which contains a record of the relative 3d positions of the upper and lower arches. These data may then form the basis for several applications, such as deriving a 4d model of the jaw motion and integrating into CAD.

In an embodiment depicted in FIG. 1B, a method 110 may further comprise the step of using 111 the surface patch files to design a dental prosthetic. Dental prosthetics designed based on only a single 3-dimensional position of an individual's jaw often require manual reconfiguration to accommodate interference from teeth due to the unique jaw motion of the individual. By animating 3-dimensional models of the individual based on that individual's actual jaw motion, most (if not all) reconfiguration of a prosthetic designed using the animation can be eliminated.

### IMAGING APPARATUS

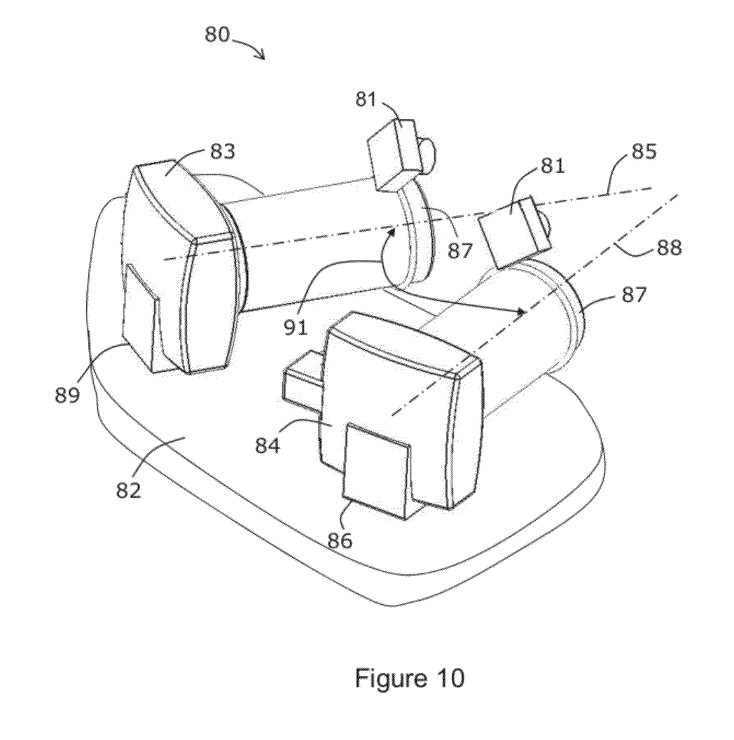

The present invention may be embodied as an imaging apparatus 80 for obtaining a time-based set of image groups of arches during jaw motion (see, e.g., FIG. 10). The imaging apparatus 80 comprises a frame 82 to which a first imaging device 84 is attached. The first imaging device 84 is capable of capturing images within a first field of view. The first field of view is generally centered about an optical axis 88 that is typically perpendicular to an imaging plane of the first imaging device 84. The first imaging device 84 is attached to the frame 82 at a first location 86.

The imaging apparatus 80 further comprises a second imaging device 83 attached to the frame 82 at a second frame location 89 which is a fixed distance from the first location 86. The second imaging device 83 is capable of capturing images within a second field of view. The field of view of the second imaging device 83 is generally centered about an optical axis 85. The imaging devices 84, 83 are oriented such that the first field of view and the second field of view overlap in the region of interest. In this configuration, the imaging devices 84, 83 are also positioned such that the angle 91 formed by their respective optical axes 88, 85 is less than approximately 30°. In this way, the imaging devices 84, 83 are configured for low-angle, image-based photogrammetric modeling. In an embodiment, the optical axes 88, 85 of the imaging devices 84, 83 are generally parallel and therefore do not form an angle with respect to each other. For the purposes of the present invention, such a parallel configuration of optical axes 88, 85 is said to be 0° and is considered to be within the scope of the invention.

In an exemplary embodiment, suitable imaging devices 84, 83 are cameras having lenses and a computer interface, e.g., a Universal Serial Bus (“USB”) communication interface. In this embodiment, three cameras, mounted at low angles to each other on the frame of the imaging apparatus, are configured to simultaneously obtain image triplets at a sampling rate of up to approximately 20 Hz (although higher rates may be possible). The cameras are provided with lenses focused at a working distance of approximately 10 cm from the front of the camera lens. An exemplary apparatus of this embodiment provides a field of view of approximately 40 mm×50 mm. A four-camera apparatus (or more) may be used for increased accuracy.

The distance between imaging devices 84, 83 and the low angles between the optical axes 88, 85 allow for compact and light-weight apparatuses that are readily hand-held.

The image apparatus 80 may further comprise a light source 81 attached to the frame 82. The light source 81 is configured to provide light (illumination) to a region of interest. The light of the light source 81 may be generally directed to illuminate the fields of view of the imaging devices 84, 83. The image apparatus 80 may further comprise one or more optical filters 87. The optical filters 87 may be configured to attenuate light at a fluorescent excitation frequency from reaching the imaging devices 84, 83. There may be one optical filter 87 which functions to filter light for all imaging devices 84, 83, or each imaging device 84, 83 may have a respective optical filter 87.

In embodiments where the imaging apparatus 80 is configured to obtain image sets of fluorescent subject matter, the light source 81 provides light at an excitation frequency and the fluorescent subject matter emits light at an emission frequency. The optical filters 87 may be configured to attenuate light at the excitation frequency from the light reaching the image devices 84, 83. In another embodiment, the optical filters 87 are configured to pass only light at the emission frequency (approximately). Further detail is provided in the following exemplary embodiment.

### EXEMPLARY EMBODIMENT

FIG. 8 illustrates the relation between three digital cameras of an exemplary imaging apparatus 600. A center digital camera 602, left digital camera 604, and right digital camera 606 are shown. The optical axes of the three cameras are shown as 610, 612, and 608, respectively. The optical axes 610, 612, 608 pass through the center of the imaging sensor for each digital camera. The angle 616 between the center and left cameras 602, 604 is less than 30°, as well as the angle 614 between the center and right-side cameras 602, 606. As a result, image pairs produce by these pairs of cameras (center-left; center-right) may be used to determine two 3d point meshes according to the methods of this invention. The angle 624 between the left and right-side cameras 604, 606 is greater than 30°. As such, even though a third pair of images may provide increased accuracy by yielding another 3d point mesh, the image pair produced by the left and right-side cameras 604, 606 is not suitable for the methods of this invention.

An angle 622 between a right midline 618 (between center and right cameras 602, 606) and a left midline 620 (between center and left cameras 602, 604), representing an angle 622 between the two useable pairs of images, may be greater than 30°. Increased accuracy may be achieved using additional image pairs, as long as the angle between each image of any pair is useable (i.e., less than 30°).

In an embodiment, the optical axes 608, 610, 612 are generally parallel and therefore do not form an angle with respect to each other. In this disclosure, such a parallel configuration of optical axes is said to be 0° and is considered to be within the scope of the invention.

FIG. 9 illustrates a front view configuration of a self-contained imaging apparatus 700 having a plastic enclosure 701. The lenses of the three digital cameras 602, 604, 606 are shown. Five blue LEDs 702 are used to provide excitation light for fluorescent texture(s) (applied according to the methods disclosed herein). Three laser diodes 704 provide alignment beams to assist a user with positioning the imaging apparatus 700. A pushbutton 706 is used to turn on both the laser diodes 704 and the blue LEDs 702. Once the imaging apparatus 700 is positioned by the user, pushbutton 708 is used to begin the recording of image triplets. Indicator light 710 illuminates to show that the unit is ready to image, and indicator light 712 illuminates during recording.

In an embodiment, the imaging apparatus 700 is configured to obtain image triplets at a rate of up to approximately 20 Hz (although higher rates may be possible). The cameras 602, 604, 606 are each provided with lenses focused at a position on the respective optical axis 608, 610, 612 at a distance of approximately 10 cm from the front of the respective camera. An exemplary imaging apparatus of this embodiment provides a field of view of approximately 40 mm×50 mm. A four-camera apparatus (or more) may be used for increased accuracy.

The distance between the imaging devices and the low angles between the optical axes allow for compact and light-weight apparatuses that are readily hand-held. When obtaining images of textured surface regions, the three cameras should have an adequately large amount of overlap between their fields of view.

In embodiments where the imaging apparatus is configured to capture image groups of fluorescent subject matter, the light source 702 provides light at an excitation frequency and the fluorescent subject matter emits light at an emission frequency. In an exemplary embodiment, the image devices may be fitted with optical bandpass filters that only pass the fluorescing (emission) wavelengths. In the case of imaging a texture based on fluorescein, the optical filters may pass, for example, wavelengths from 500 to 600 nm. In this way, the blue excitation light provided by a light source is blocked and only the green emitted light from the texture is imaged. In addition, the alignment laser diodes 704 may emit red light at 623 nm which may also be blocked by the optical filter.

When using imaging devices that detect in monochrome only, the fluorescing texture, illuminated and filtered as described above, will result in an image of a white texture against a dark background.

Non-Limiting Example of Imaging Apparatus

Light source: blue LED, 450 nm.

Cameras: 2 megapixel monochrome digital

Lens: 16 mm focal length

Optical filters: 500-600 nm bandpass filter (e.g., those available from Semrock Inc., Rochester, N.Y.)

Field of View: approximately 40×50 mm at the working distance

Working distance (to image plane): 10 cm

A large imaging volume of this exemplary apparatus is another beneficial feature of this invention. Imaging volume is the 3d region of space that can be imaged, which approximately comprises of the X and Y dimensions at the working distance times the total depth of field in the Z (axial) direction. The effective imaging volume is sufficiently large so to be able to capture both the upper and lower teeth when the mouth is opened.

The 40×50 mm field of view allows images of open positions of the mouth to be obtained. Operating at f/11 provides a depth of field of approximately +/−25 mm at the working distance. This allows images of regions of the arch with curvature to be obtained such as from positions perpendicular to the cuspids. The large imaging volume also allows images to be obtained from the front of the mouth to better capture sideways jaw motions.

An alignment system, such as a laser alignment system, may be used to assist the user with positioning the unit with respect to the patches. Lasers used in such a system may emit red radiation at 635 nm which is blocked by the camera's bandpass filter.

While monochrome cameras are generally used for photogrammetry applications, color imaging devices may also be used. Color cameras provide the ability to differentiate upper and lower textures using different colors. This can be an advantage for automated data processing.

The cameras may be triggered simultaneously to obtain image triplets at about 10 Hz. Data may be transferred from the image apparatus by, for example, USB, wireless, etc.

### EXEMPLARY METHOD—PRODUCTION OF 4D DATA

Texture

Texture is considered to be a randomized optical texture on an imaging sensor produced by an applied surface treatment on a region of the oral cavity.

In a preferred embodiment, fluorescent microspheres may be applied to the teeth as part of a composition suitable for brush-on application. The composition comprises a biocompatible volatile carrier component, for example, ethanol. The composition further comprises a polymer mixed with the carrier. Suitable polymer solutes include biocompatible polymers such as polyvinylpyrolidone (PVP), cellulose derivatives, polyvinyl alcohol, and polyethylene glycol.

The texture's features can vary spatially and in intensity. Any imaging element capable of providing a fine random pattern on an imaging sensor may be suitable.

Microsphere Carrier Composition

Obtaining images of tooth enamel presents special difficulties, since enamel is fluorescent. When excited by ultraviolet or blue light, enamel emits strongly in the yellow/green region. A beneficial feature of the carrier is color. A colored film provides a dark background for imaging the imaging elements. The dark background provides the high contrast needed to create suitable optical texture. The fluorescent methods of this invention do not work well on tooth enamel without a colored film. For the fluorescent methods in this invention, the film may be black, red, or blue, or other colors which enhance the contrast between the imaging elements and the background as viewed by the imaging devices.

Non-Limiting Example Carrier Composition:

5% Polyvinyl pyrrolidone in 90% ethanol

1% Erioglacine—blue color

0.2% Sodium dodecyl sulfate

Imaging element (microsphere) concentration approximately 10,000/ml

The colored film provides the following functions:

1. Absorbs the green light emitted by the beads, providing contrast to the beads against the background.

2. Absorbs fluorescent emission from the underlying enamel. Blue incident light enters the enamel at the perimeter of the patch and causes the enamel under the patch to fluoresce.

3. Blocks incident blue light from entering the enamel surface between the beads within the patch.

4. Does not reflect any green light emitted by the blue leds back to the cameras. All leds emit a small fraction of broad spectrum light, including green, which can reduce the contrast between the background and the beads.

The carrier may be formulated to be sufficiently volatile so as to form a film in a short period of time (e.g., less than 10 seconds). The composition may also be sufficiently viscous to minimize flow during the drying period.

The method thereby provides for the imaging of fluorescent microspheres placed on tooth surface without tooth transparency interference. The captured images may be transferred in real-time to a computer for storage and further processing.

Microsphere Application Methods

FIG. 5 shows a typical (40×50 mm) field of view, 320, for the imaging apparatus in an embodiment. An upper patch 340 and lower patch 360 are also shown. Each patch 340, 360 extends about halfway from the gingival margin to the occlusal tip of the teeth. This avoids overlapping of the patches 340, 360 when the teeth of each arch are together. The patches 340, 360 may extend onto the soft tissue about an equal distance. This application scheme provides significant curvature to the patch for improved registration. In general, textures are applied to surface regions with curvature such as the gingival margin.

A non-interfering cheek retractor may be used to keep the lips apart during application of the textures.

A flat nylon brush may be used to apply the composition to the teeth or soft tissue. When the composition is applied, the carrier evaporates leaving the microspheres trapped on the surface region under a thin polymer film. While the carrier is evaporating, the imaging elements settle by gravity to effectively result in direct contact with the surface region.

The individual may be positioned such that the surface regions to be used are generally horizontal when applying the texture. This provides good access for coating and provides a gravitational component towards the facial aspect of the surface regions. After the film dries, for example, in approximately 10 seconds, the individual may be returned to a normal, upright posture.

Microspheres are applied at a concentration to give effective texture, which is generally 50-75% of total theoretical area coverage. Additional, non-imaging imaging elements not visible to the imaging devices may be added to provide a more uniform distribution of visible imaging elements in the region. These non-visible imaging elements may be non-fluorescent and/or of a different diameter, and/or otherwise not visible to the imaging devices.

In some embodiments, textures may be placed only on the soft tissue. Soft tissue provides a naturally dark background, which allows the use of bead compositions without color.

In another embodiment according to the invention, the imaging elements applied to the upper arch may be a different color than those applied to the lower. Using color imaging devices with such differently colored imaging elements provides a way to differentiate the upper and lower dentition during image analysis, which can enhance automatic data processing.

The patches are located on the anatomy to be animated. 4d captures of more than one region of the mouth may be obtained, with the results being coordinated and combined.

In an embodiment, the texture may be applied to a premolar, cuspid, and a lateral incisor. Images may be obtained from an aspect generally perpendicular to the cuspids. This provides a field with significant dimension in orthogonal directions.

In another embodiment, the texture may be applied as printed, water-transferable film that contains imaging elements printed in a prescribed pattern. Such imaging elements may be printed small dots (e.g., from 0.001 to 0.005 inches diameter) having a random pattern. The printed pattern may be fluorescent. The background may also be black or printed black to enhance the contrast of the pattern.

Clinical Imaging

FIG. 6, 400 shows a single image (one of a triplet of images) of 35 micron diameter fluorescent microspheres in the mouth as described. A tooth 402, outside of the patch area, is visible due to fluorescence of the enamel. The upper patch 404 and lower patch 406 show the random texture provided by the imaging elements.

The main steps in clinical imaging include, without limitation:
1. Use a cheek retractor to keep the lips apart;
2. Apply texture to the oral anatomy;
3. Obtain images and image groups; and
4. Wash texture off using water.

A hand-held imaging apparatus is used from a distance of approximately four inches, to image the patches. Clinical imaging involves capturing a time-based set of image groups of the patches. Triggering the cameras at, for example, 10 Hz produces ten image groups per second, or, once processed, ten 3d jaw positions per second. Each individual image captures both upper and lower patches. The set of image groups may be obtained over about 10-15 seconds.

Using the methods of the present invention, clinical jaw motion imaging is performed with the lips held apart, and the methods of this invention have little to no interference with natural jaw motion. Captured clinical jaw motions may include: border movements, random actions, chew cycles, clenching, and open/close. The motions to be captured may depend upon the specific application.

In an exemplary application, where a new prosthetic tooth is to be designed, example jaw motions include:

1. Protrusion—these data may be used to assist with designing the anterior guidance needed to ensure disclusion of the posterior teeth.

2. Open/close—a true arc-of-closure may be obtained and used to ensure the designed tooth hits the opposing fossa close to the bottom in a balanced fashion.

3. Random chew-in motion—this motion may be used to create a dynamic surface, representing the locus of positions assumed by antagonist teeth. Designing a new tooth against this surface ensures that the tooth will not interfere when placed in the mouth.

Deriving Surface Patch Files

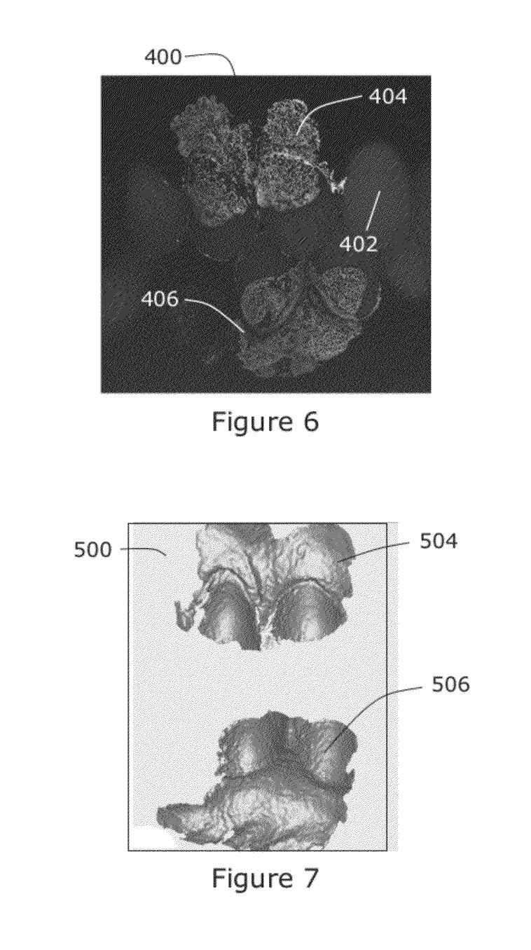

A surface patch file is a 3d digital representation containing both upper and lower patches (see, e.g., FIG. 7). These data comprise the relative 3d position of the arches at a single time point.

Commercially available photogrammetry software (e.g., PhotoModeler™ from EOS Systems of Vancouver, BC Canada) may be used to analyze the texture within the patch images and accurately produce 3d surface patch files. PhotoModeler™ is software used to perform photogrammetry on multiple photographs of the same field taken at various angles.

Producing a 3d point mesh from a group of images requires characterization of the optical and physical system. The process may be considered in two steps. The following actions may be performed by the PhotoModeler™ software:

1. System Setup

a. Camera calibration—the individual imaging devices (e.g., cameras, etc.) are calibrated to derive lens correction parameters used to adjust images for accurate subpixel marking.

b. Camera orientation—the location on each image sensor of common features of the images are identified. In one embodiment, the software can automatically identify such features as “SmartPoints™ ” (Eos Systems). About 300 SmartPoints™ may be identified. These points are used to obtain the orientation of the cameras in 3d space.

c. Scale—may be added by obtaining an image group that includes a calibration target having a set of known distances. The distance between the cameras may then be determined using PhotoModeler™. These values are unique for each 3-camera imaging apparatus. The distances between the cameras may then be used to provide scale to the already oriented system.

2. Dense Surface Modeling

Dense surface modeling (“DSM”) is a set of image-based search algorithms that look for pixel-level groupings of texture that look alike between a pair of low-angle images. This may be done in a regular grid-like manner, and a plurality of 3d locations is computed. DSM can be used to obtain uniform surface measurements that would not be practical with other techniques such as point-based photogrammetry.

Once the system setup has been adequately defined, DSM may be performed. The DSM algorithm uses pairs of images to derive a point mesh. Pre-defined areas of one image are searched for matching locations using an n×m patch of imagery from the paired image. Matches are optimized and recomputed on a sub-pixel level. The matched orientations on each camera's imaging sensor are then used to create 3d point locations (as a point mesh) using the camera position and scale information.

For a 3-camera imaging apparatus described in this disclosure, two low-angle image pairs are used: one between the center and left cameras, and a second between the center and right cameras. The two point meshes produced from the two pairs of images are registered to each other and merged into a single point mesh having definable point spacing. The final point mesh of the upper and lower textured surface regions may then be saved as a surface patch file in any file format such as those known in the art.

As previously described, a captured jaw motion sequence may be represented by a set of surface patch files. Each 3d surface patch file in a sequence may be considered an individual “frame,” similar to the 2-dimensional image frames of a common video sequence. Sampling at 8 Hz, a three-camera imaging apparatus would produce three simultaneous images per image group (sample) and eight image groups per second, for a total of 24 images per second. A ten second clinical sequence (240 camera images) may comprise of eighty 3d positions.

These data provide an accurate record of the relative position of the arches at points in time and are suitable for registration to other surface anatomy obtained by known means.

### MODELING

The manipulation of time-based 3d datasets is well known in the art. An example is hereby given of a modeling method for utilizing the surface patch files produced by this invention.

Reference Position

In an embodiment, one of the surface patch files in a sequence is defined as a “reference frame” for deriving relative motion expressions. Any 3d surface patch file in a motion sequence may serve as a reference.

Design Anatomy

Design anatomy, or more complete 3d models of the oral anatomy, is the anatomy required for the design of a particular dental prosthetic. Design anatomy includes the surface that underlies and extends from the upper and lower textural surface regions. These data are typically obtained as 3d point mesh files using well known methods such as 3d intraoral scanning, or scanning of dental casts or impressions. Such files generally have uniform point spacing. The surface patch files produced by the methods of this invention lie within the design anatomy.

Deriving Expressions to Characterize Motion

The upper and lower design anatomy may be registered to the reference frame surface patch file to produce an enhanced reference frame. This is done to create the maximum usable surface area for the subsequent registrations used to build the 4d model.

The upper surface patch data in another frame (frame n) are then registered to the extended upper data in the enhanced reference frame. The upper data in frame n now coincides with the corresponding upper data of the reference frame, and the lower data are ‘displaced’ from the position of the corresponding extended lower data of the reference frame. This displacement may be expressed as a transform function, derived by registering the reference lower data to the displaced (frame n) lower data from another time frame. The transform thereby produced for frame n expresses the coordinate system shift required to move the lower data from its position in the reference frame to its position in frame n.

Repeating these steps for the remaining frames in a 4d sequence produces a set of transforms that describe the incremental change in position of the lower arch with respect to a fixed upper for the sequence. These time-based transform data also constitute a 4d model. The transforms may then be used to animate the design anatomy. Other mathematical schemes and technical approaches may be used to achieve similar results.

Since the surface derived from the microspheres may lie just off the true oral surface, the design anatomy may be shelled to assist with the registration of surface patch files. Alternatively, the surface patch files may be shrunk by a similar amount.

The incremental displacement of the upper and lower arches from one time point to the next is usually not large compared with the size of the patches. Therefore, after registering the reference frame to the design anatomy for determining a transform, the remaining surface patch files in a sequence may be registered and analyzed automatically.

Animation of Design Anatomy

The set of transforms derived for a particular motion sequence may be used to animate the design anatomy. The transforms and the design anatomy may be integrated into CAD to provide patient specific motion. Animation and display may be accomplished by a variety of means and still be within the scope of this invention.

Integration into CAD

When designing a new tooth, for example, the new tooth may be animated against its antagonist design anatomy. The CAD tools used to shape new teeth may then be used to design the tooth based on the contact and interferences observed during the animation.

In other embodiments, the surface patch files may be registered and used to animate 3d anatomic data produced by other modes of imaging such as, for example, x-ray, ultrasound, or magnetic resonance imaging.

Other Exemplary Applications

Applications include the design of any prosthetic or oral appliance that requires occlusion of the teeth, such as crowns, bridges, dentures, and removable oral appliances. Other application areas include diagnostics and surgical applications.

For enhanced crown design, the design oral anatomy may refer to the anatomy associated with the design for a new tooth (teeth), adjacent teeth, and antagonist teeth. For example, animating the occlusal surface of antagonist teeth provides a method of generating a dynamic surface (similar to a chew-in random motion) which represents the locus of antagonist tooth positions. Designing crowns against this dynamic surface reduces the interferences when the crown is fitted in the mouth.

Specific jaw motions such as chew cycles, open/close, and border movements may be used to optimize the occlusion during the design of a new tooth.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A method of tracking jaw motion, the jaw having an upper arch and a lower arch, the method comprising the steps of:
applying a texture to one or more surface regions of the upper arch and one or more surface regions of the lower arch;
obtaining a set of at least two image groups of the applied texture using an extraoral imaging device, wherein the images of each image group are obtained simultaneously and each image of each image group is obtained along an optical axis which is less than 30° with respect to an optical axis used to obtain another image of the same image group, and wherein each image group is obtained at a different time during jaw motion; and
using image-based registration to produce a time-based set of at least two 3-dimensional point meshes of the applied texture, wherein each point mesh is produced using one image group of the set of image groups.

2. The method of claim 1, wherein each image group comprises three images.

3. The method of claim 1, further comprising the step of using the produced time-based set of 3-dimensional point meshes to design a dental prosthetic.

4. The method of claim 1, wherein the texture comprises a plurality of imaging elements.

5. The method of claim 4, wherein the imaging elements are fluorescent microspheres.

6. The method of claim 1, wherein the texture is applied using a water transfer decal.

7. The method of claim 1, wherein the image groups are obtained at a rate of approximately 10 Hz.

8. The method of claim 1, wherein the step of producing the time-based set of at least two 3-dimensional point meshes comprises the sub-steps of:
searching two images from an image group to locate common textural features, wherein each textural feature is a portion of the pair of images having corresponding textures;
computing a 3-dimensional location of each common textural feature; and
assembling the 3-dimensional locations to produce a 3-dimensional point mesh corresponding to the two searched images.

9. The method of claim 8, wherein each image group comprises more than two images and further comprising the sub-steps of:
repeating the sub-steps of claim 8 until each image of the image group has been searched at least one time;
merging the 3d point meshes to produce a single 3d point mesh corresponding to the image group.

10. The method of claim 1, wherein the step of applying a texture, comprises the sub-steps of:
applying a texture having a first optical characteristic to one or more surface regions of the upper arch; and
applying a texture having a second optical characteristic to one or more surface regions of the lower arch, wherein the second optical characteristic is different from the first optical characteristic.

11. The method of claim 10, wherein the optical characteristic is color.

12. An apparatus for obtaining two or more low angle image groups of a portion of a jaw, comprising:
a frame;
a first electronic imaging device attached to the frame at a first frame location, the first image sensor having an optical filter and oriented to obtain an image along a first optical axis;
a second electronic imaging device attached to the frame at a second location, the second image sensor having a second optical filter and oriented to obtain an image along a second optical axis;
wherein the first frame location and the second frame location are at a fixed distance from each other and the imaging devices are configured such that the first optical axis and the second optical axis are at an angle of less than 30° with respect to each other.

13. The apparatus of claim 12, wherein the electronic imaging devices are oriented such that the first optical axis and the second optical axis are substantially parallel to one another.

14. The apparatus of claim 12, further comprising at least one light source attached to the frame and configured to provide light to a region of interest.

15. The apparatus of claim 14, wherein the optical filters are configured to reduce the amount of light from the light source that reaches the respective imaging devices.

16. The apparatus of claim 11, further comprising a range finder attached to the frame.
